(19) United States
(12) Patent Application Publication
(10) Pub. No.: US 2022/0160986 A1
(43) Pub. Date: May 26, 2022

(54) WEARABLE BREATHING TUBE SYSTEM AND BREATHING EQUIPMENT WITH THE SAME

(71) Applicant: SHANGHAI ASCLEPIUS MEDITEC CO., LTD., SHANGHAI (CN)

(72) Inventor: HSIN-YUNG LIN, TAOYUAN CITY (TW)

(21) Appl. No.: 17/595,821

(22) PCT Filed: May 25, 2020
(86) PCT No.: PCT/CN2020/091979

§ 371 Date: Nov. 24, 2021

(30) Foreign Application Priority Data
May 28, 2019 (CN) 201910448642.0

(51) Int. Cl. A61M 16/08; A61M 16/00; A61M 16/14

(52) U.S. Cl. CPC A61M 16/0875; A61M 16/0808; A61M 16/0087; A61M 2209/088; A61M 16/0833; A61M 2202/02; A61M 16/14

(57) ABSTRACT

A breathing tube system includes a gas receiving tube, an output tube and a flame arrester. The gas receiving tube is configured to receive a breathing gas. The output tube is coupled to the gas receiving tube to form a supply pipeline. The output tube is configured for a user to wear and to output the breathing gas from the supply pipeline to the user. The flame arrester is configured in the supply pipeline. Accordingly, the safety of the breathing equipment could be improved.

### BACKGROUND OF THE INVENTION

### 1. Field of the Invention

The present invention relates to a breathing equipment, especially to a breathing equipment including a breathing tube system with a flame arresting function.

### 2. Description of the Prior Art

As people have been paying much attention on health developments, many developments in medical technology are often targeted on treating diseases to prolong human life. Most of the treatments in the past are passive; namely, diseases are treated only when they occur. The disease treatments include operations, medication treatments, radiation therapies, or even medical treatments for cancer. However, in recent years, most of the researches from medical experts are gradually moved towards the preventive medical methods, such as healthy food, screening and the prevention of inherited diseases, to actively prevent diseases from occurring in the future. Moreover, for the prolongation of human life, many anti-aging and anti-oxidation technologies including skin care products and anti-oxidation food/medicine are gradually developed and adopted by the general public.

It has been found that there are instable oxygen species (0+), also known as free radicals, in the human body. The free radicals are usually generated because of diseases, diet, environment and one's lifestyle, and can be excreted in the form of water by reacting with the inhaled hydrogen. With this method, the amount of free radicals in the human body can be reduced, thereby restoring the body condition from an acidic state to an alkaline state, achieving an anti-oxidation, anti-aging and beauty health effect, and even eliminating chronic diseases. Furthermore, there are also clinical experiments showing that the patients who need to inhale the high concentration oxygen gases for an extended time would experience lung damage, but this situation could be ameliorated by inhaling hydrogen.

A conventional method of inhaling hydrogen is that a user wears the breathing mask, which is connected to a hydrogen generating device for transferring the hydrogen gas generated by the hydrogen generating device, to inhale the hydrogen gas. However, the gas outputted from the hydrogen generating device may contain unnecessary and excess water vapor such as additional water vapor for preventing excessive drying of the gas outputted by the hydrogen generating device, water vapor generated by the mouth and nose of the user during breathing, and water vapor generated by the temperature change caused by the user's breathing. If the gas inhaled by the user contains excess water vapor, it will be difficult for the user to inhale the gas smoothly, thereby losing the motivation of inhaling the gas.

In addition, if the hydrogen gas in the pipe is accidentally ignited because of static electricity while the user inhales the hydrogen by the breathing mask, the ignited hydrogen may enter the user's respiratory tract from the breathing mask and cause the risk concerns of personal safety.

### SUMMARY OF THE INVENTION

Therefore, one category of the present invention is to provide a breathing tube system to solve the problems of the prior art.

According to an embodiment of the present invention, the breathing tube system includes a gas receiving tube, a first connecting tube, an output tube and a flame arrester. The gas receiving tube is configured to receive a breathing gas which can be inhaled by a user. The first connecting tube is coupled to the gas receiving tube and the output tube. The user can wear the output tube to inhale the breathing gas. Wherein, the gas receiving tube, the first connecting tube and the output tube form a supply pipeline, and the flame arrester is configured in the supply pipeline to block the flame spread of flammable gas and flammable liquid vapor.

Wherein, the breathing tube system further includes a catchment canister coupled to the gas receiving tube and the first connecting tube. The catchment canister is configured to collect a liquid in the supply pipeline. Wherein, the diameters of the gas receiving tube and the first connecting tube are smaller than the maximum inner diameter of the catchment canister.

Wherein, the catchment canister has a first canister body and a second canister body which can be separated from each other, and a catchment canister sealing ring is configured between the first canister body and the second canister body to couple the first canister body to the second canister body.

Furthermore, the breathing tube system further includes a first switch coupled to the first connecting. The catchment canister is separably coupled to the gas receiving tube and the first switch.

Wherein, the flame arrester is configured between the catchment canister and the first connecting tube.

Furthermore, the flame arrester is contained in the first switch and coupled to the catchment canister by the first switch.

Wherein, the output tube has two gas outlets to output the breathing gas to the user, and the first connecting tube includes a pair of sub-connecting tubes respectively coupled to a side opening of the output tube.

Another one category of the present invention is to provide a breathing equipment to solve the problems of the prior art.

According to an embodiment of the present invention, the breathing equipment includes a breathing tube system. The breathing tube system further includes a gas receiving tube, a first connecting tube, an output tube and a catchment canister. The gas receiving tube is configured to receive a breathing gas which can be inhaled by a user. The first connecting tube is coupled to the gas receiving tube and the output tube. The user wears the output tube of the breathing tube system to inhale the breathing gas. Wherein, the gas receiving tube, the first connecting tube and the output tube of the breathing tube system form a supply pipeline. The catchment canister is coupled to the supply pipeline for collecting a liquid in the supply pipeline.

Wherein, the diameter of the supply pipeline is smaller than the maximum inner diameter of the catchment canister.

Wherein, the breathing tube system further includes a first switch configured to couple the first connecting tube to the catchment canister.

Furthermore, the first connecting tube includes a pair of sub-connecting tubes, and the sub-connecting tubes are coupled to the catchment canister by the first switch.

Wherein, the catchment canister is configured between the gas receiving tube and the first connecting tube, and the breathing tube system further includes a flame arrester configured between the catchment canister and the first connecting tube.

Wherein, the breathing equipment further includes an electrolytic cell configured to electrolyze the liquid collected by the catchment canister and generate the breathing gas.

Wherein, the breathing equipment further includes an atomizer configured to generate an atomizing gas from the liquid collected by the catchment canister and to mix the atomizing gas with a source gas to form the breathing gas.

In summary, the flame arrester is provided in the breathing tube system of the present invention to reduce the possibility of gas ignition to hurt the user. On the other hand, the catchment canister is provided in the breathing tube system of the present invention to collect the liquid in the supply pipeline, thereby reducing the discomfort of the user while inhaling breathing gas. Furthermore, the liquid in the catchment canister can be used to assist in generating breathing gas or to clean the gas pipeline of the gas generating device. Compared with the prior art, the present invention has the advantages of increasing the safety and the comfort of the user, and improving the efficiency of the equipment.

### BRIEF DESCRIPTION OF THE APPENDED DRAWINGS

FIG. 1 is a function block diagram illustrating a breathing equipment in an embodiment of the present invention.

FIG. 2 is a schematic diagram illustrating a breathing tube system in another embodiment of the present invention.

FIG. 3 is a schematic diagram illustrating the breathing tube system in another embodiment of the present invention.

FIG. 4 is a schematic diagram illustrating the breathing tube system in another embodiment of the present invention.

FIG. 5 is a function block diagram illustrating the breathing equipment in another embodiment of the present invention.

FIG. 6 is an exploded diagram illustrating the catchment canister in the FIG. 2.

FIG. 7 is an exploded diagram illustrating the breathing tube system in another embodiment of the present invention.

### DETAILED DESCRIPTION OF THE INVENTION

For the sake of the advantages, spirits and features of the present invention can be understood more easily and clearly, the detailed descriptions and discussions will be made later by way of the embodiments and with reference of the diagrams. It is worth noting that these embodiments are merely representative embodiments of the present invention. However, it can be implemented in many different forms and is not limited to the embodiments of the present invention or corresponding embodiments. Rather, these embodiments are provided so that this disclosure will be thorough and complete.

The terms used in the various embodiments disclosed of the present invention are only used to describe specific embodiments, and are not intended to limit the various embodiments disclosed of the present invention. As used herein, the singular form also includes the plural form unless the context clearly indicates otherwise. Unless otherwise defined, all terms (including technical and scientific terms) used in this specification have the same meanings as commonly understood by one of ordinary skill in the art to which the various embodiments disclosed herein belong. The above terms (such as those defined in commonly used dictionaries) will be interpreted as having the same meaning as the contextual meaning in the same technical field, and will not be interpreted as having an idealized or overly formal meaning, Unless explicitly defined in the various embodiments disclosed herein.

In the description of this specification, the description with reference to the terms “an embodiment”, “a specific embodiment”, etc. means that a specific feature, structure, material, or characteristic described in conjunction with the embodiment is included in at least one embodiment of the present invention. In this specification, the schematic expressions of the above terms do not necessarily refer to the same embodiment. Furthermore, the particular features, structures, materials, or characteristics described may be combined in any suitable manner in any one or more embodiments.

In the description of the present invention, unless otherwise specified or limited, it should be noted that the terms “coupled”, “connected”, and “setup” should be understood in a broad sense. For example, they may be mechanically or electrically connected, may be connected directly, also may be connected by an intermediate medium. For those skilled in the art, the specific meanings of the above terms can be understood according to specific situations.

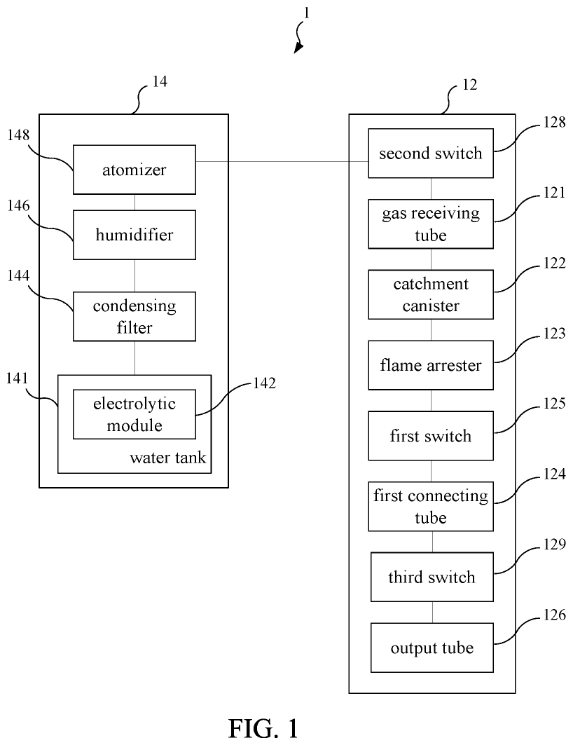

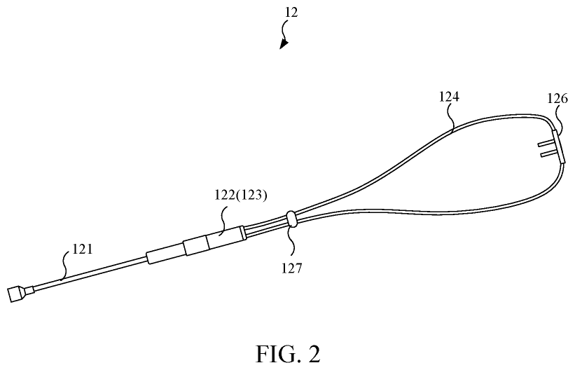

Please refer to FIG. 1 and FIG. 2. FIG. 1 is a function block diagram illustrating a breathing equipment 1 in an embodiment of the present invention. FIG. 2 is a schematic diagram illustrating a breathing tube system 12 in another embodiment of the present invention. As shown in FIG. 1 and FIG. 2, the breathing equipment 1 includes the breathing tube system 12, and the breathing tube system 12 further includes a gas receiving tube 121, a flame arrester 123 and an output tube 126. The gas receiving tube 121 is configured to receive a breathing gas which can be inhaled by a user. The output tube 126 is provided for the user to wear and coupled to the gas receiving tube 121 to form a supply pipeline. The output tube 126 is configured to output the breathing gas from the supply pipeline to the user. The flame arrester 123 is configured at the place where the breathing gas flows in the breathing tube system and can be configured in the supply pipeline to reduce or block the spread of flammable gas or flammable liquid vapor to the output tube 126.

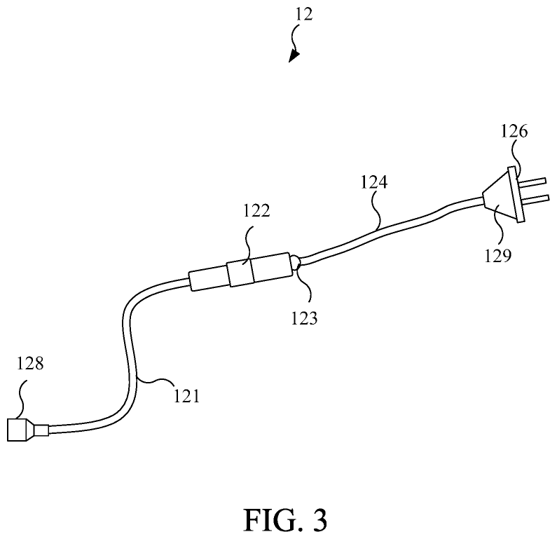

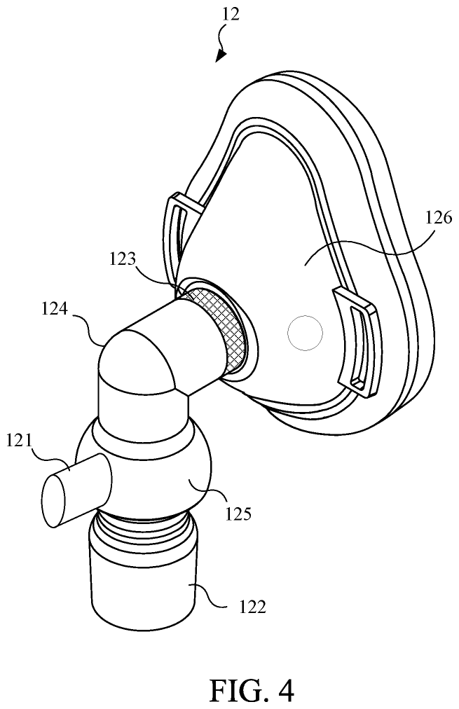

Please refer FIG. 2 to FIG. 4. FIG. 3 is a schematic diagram illustrating the breathing tube system 12 in another embodiment of the present invention. FIG. 4 is a schematic diagram illustrating the breathing tube system 12 in another embodiment of the present invention. The breathing tube system 12 can be a nasal cannula as shown in FIG. 2. The output tube 126 has multiple gas outlets corresponding to the user's respiratory tract. Furthermore, the gas outlets of the output tube 126 may correspond to any one of the user's nose, mouth or a combination thereof. The supply pipeline between the flame arrester 123 and the output tube 126 can be a plurality of first connecting tubes 124 (such as a pair of sub-connecting tubes) to be respectively coupled to the corresponding gas outlets. The first connecting tubes 124 of the breathing tube system 12 are girdled together by the girdle 127 for the user to wear. However, the breathing tube system of the present invention is not limited to the aforementioned forms, and other structures suitable to be worn by the user and provide the breathing gas for the user to inhale can be adopted. For example, the breathing tube system 12 shown in FIG. 3 is in the form of a single first connecting tube 124 between the flame arrester 123 and the output tube 126, or the output tube 126 of the breathing tube system 12 shown in FIG. 4 is in the form of a mask.

However, regardless of the forms of the breathing tube system 12 worn by the user, the breathing circuit system 12 has a supply pipeline coupled to the gas receiving tube 121 and the output tube 126 to provide breathing gas for the user to inhale. Since the breathing gas may include ignitable gas (such as hydrogen), the flame arrester 123 of the present invention can be configured at any portion of the supply pipeline to reduce or prevent ignited gas from spreading to the output tube 126 worn by the user to hurt the user. In practice, the flame arrester 123 can be configured in the gas receiving tube 121, on the output tube 126 and any portion of the connecting channels between the gas receiving tube 121 and the output tube 126. Furthermore, the flame arrester 123 can be formed as one part of the connecting channel between the gas receiving tube 121 and the output tube 126. In other words, the breathing gas from the gas receiving tube 121 flows through the flame arrester 123 to avoid the risk of the breathing gas spreading to the output tube 126 after being ignited. Moreover, in order to reduce the possibility of ignited gas spreading outward, the flame arrester 123 can be configured at the output tube 126, which is closest to the user, of the breathing tube system 12. As shown in FIG. 4, the flame arrester 123 is configured at a side of the output tube 126 near the first connecting tube 124. Since the flame arrester 123 is configured on the output tube 126, which is closest to the user, of the breathing equipment 1, the total gas between the flame arrester 123 and the user is efficient decreased, thereby reducing the possibility of gas ignition between the flame arrester 123 and the user to hurt the user.

Furthermore, an acceleration component with a special structure can be configured in the supply pipeline of the breathing tube system 12 to increase the flow speed of the breathing gas by flowing through the acceleration component. For example, the flow speed of the breathing gas can be increased according to the Bernoulli's principle. In practice, the mentioned acceleration component can be the aforementioned flame arrester. The gas pressure of the front end of the flame arrester may be greater than that of the rear end of the flame arrester in the supply pipeline. The flow speed of the breathing gas is increased when the gas pressure of the flame arrester is released when the breathing gas flows through the flame arrester. At this time, since the breathing gas moves fast enough after flowing through the flame arrester, the breathing gas in the breathing tube system 12 can still be effectively inhaled by the user but not easily spill to the external environment, even if the output tube 126 in FIG. 2 only has an opening structure corresponding to the user's nose. In other words, the flame arrester also has the effect of improving the efficiency of the user inhaling the breathing gas.

On the other hand, in order to reduce the difficulty of breathing for the user, the breathing tube system 12 of the present invention further includes a catchment canister 122 configured to collect a liquid in the supply pipeline. In detail, the catchment canister 122 is configured to collect the liquid in the supply pipeline formed between the gas receiving tube 121 and the output tube 126. In practice, because the breathing gas received by the gas receiving tube 121 may comprise excess water vapor, it may cause the user to choke if the user directly inhales the breathing gas comprising excess water vapor, thereby losing the motivation to inhale the breathing gas. Therefore, the catchment canister 122 collects the excess water vapor in the supply pipeline to reduce the possibility of the user choking on the water vapor. However, the liquid contained in the supply pipeline is not limited to the aforementioned form. In practice, the liquid may be formed by humidified breathing gas, water vapor exhaled by the user during breathing, or water vapor generated by the temperature change caused by the user's breathing or gas pressure change.

Please refer FIG. 2 to FIG. 4. As shown in FIG. 3, in one embodiment, the catchment canister 122 can be configured between the gas receiving tube 121 and the first connecting tube 124. The breathing gas flows from the gas receiving tube 121, through the catchment canister 122 and the first connecting tube 124, to the output tube 126 in sequence. In other words, the catchment canister 122 can be a part of the supply pipeline. Meanwhile, the aforementioned flame arrester 123 can be configured at the position where the gas of the catchment canister 122 flows through, and can be configured between the catchment 122 and the first connecting tube 124. For example, the flame arrester 123 is configured at one side of the catchment canister 122 close to the first connecting tube 124. However, the position of the flame arrester 123 is not limited thereto. As shown in FIG. 2, the flame arrester 123 also can be a part of the catchment canister 122.

Furthermore, in order to exhaust the liquid in the catchment canister 122 effectively, the catchment canister 122 can be separably coupled to the gas receiving tube 121 and the first connecting tube 124. The catchment canister 122 can be easily separated from the gas receiving tube 121 or the first connecting tube 124, thereby exchanging the catchment canister 122 or exhausting the liquid in the catchment canister 122. Because the catchment canister 122 is coupled to the output tube 126 by the first connecting tube 124, the length of the first connecting tube 124 of the breathing tube system 12 can be adjusted to reduce the discomfort of the user wearing the output tube 126 when the catchment canister 122 is separated. However, in one embodiment, the breathing tube system 12 may not include the first connecting tube 124. At this time, the catchment canister 122 can be separably coupled to the gas receiving tube 121 and the output tube 126, so that the catchment canister 122 could be separated from the gas receiving tube 121 or the output tube 126.

In addition, the supply pipeline may comprise the liquid for a long time, and there may be a possibility of breeding bacteria in the gas receiving tube 121 or the output tube 126. Therefore, the breathing tube system 12 of the present invention further includes a switch for detachably coupling the output tube 126 and the gas receiving tube 121. In the present invention, the switch can be divided into at least a first switch, a second switch and a third switch according to their positions, and the functions or structures of these three switches can be completely the same or different. Moreover, these three switches can coexist or be arbitrarily combined in the breathing tube system in the same embodiment. As shown in FIG. 3, the third switch 129 can be configured on the output tube 126 or the first connecting tube 124, or directly formed on the output tube 126 or the first connecting tube 124, so that the output tube 126 is detachably coupled to the first connecting tube 124. As shown in FIG. 4, the breathing tube system 12 can further include a first switch 125 directly formed or configured on the first connecting tube 124, so that the gas receiving tube 121 or the catchment canister 122 can be detachably coupled to the first connecting tube 124. Wherein, the first switch 125 also can be directly formed or configured on the gas receiving tube 121 or the catchment canister 122 to be coupled to the first connecting tube 124. Therefore, the breathing tube system 12 of the present invention can easily exchange the output tube 126 with the same or different sizes according to the various users or service life, and exchange the first connecting tube 124 according to different breathing gases or the cleanliness of the supply pipeline.

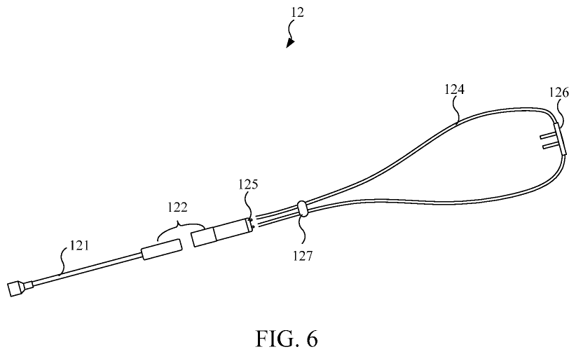

In practice, one end of the catchment canister 122 is detachably coupled to the gas receiving tube 121. The other end of the catchment canister 122 does not have to be detachably coupled to the first connecting tube 124, the first switch 125 or the output tube 126, but can still easily exhaust the liquid from the catchment canister 122. In other words, the catchment canister 122 can be integrally formed with the first connecting tube 124, the first switch 125 or the output tube 126. Similarly, the catchment canister 122 also can be integrally formed with the gas receiving tube 121, and then be detachably coupled to the first connecting tube 124, the first switch 125 or output tube 126. Please refer to FIG. 6. FIG. 6 is an exploded diagram illustrating the catchment canister 122 in the FIG. 2. In one embodiment, the catchment canister 122 may consist of a plurality of components. As shown in FIG. 6, the first component of the catchment canister 122 is coupled to the gas receiving tube 121, and the second component of the catchment canister 122 is coupled to the first switch 125. The gas receiving tube 121 is coupled to the output tube 126 when the first component and the second component of the catchment canister 122 are assembled to each other. The first component of the catchment canister 122 can be directly formed on the gas receiving tube 121, and the second component of the catchment canister 122 can be directly formed on the first switch 125.

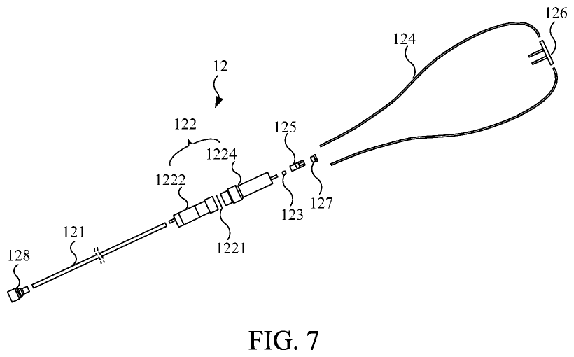

Please refer to FIG. 7. FIG. 7 is an exploded diagram illustrating the breathing tube system 12 in another one embodiment of the present invention. The gas receiving tube 121 is further coupled to a second switch 128. The second switch 128 is configured to be coupled to a gas supplying equipment which can provide the breathing gas. The second switch 128 can be a gas switch to switch the inner diameters from 15 mm to 6 mm. The outer diameter of the gas receiving tube can be 6 mm to match with the second switch. Moreover, the catchment canister 122 can be divided into a first canister body 1222 and a second canister body 1224, and the first canister body 1222 and the second canister body 1224 are coupled to each other by a catchment canister sealing ring 1221. The outer sides of the first canister body 1222 and the second canister body 1224 respectively have an opening configured to be coupled to the gas receiving tube 121 or the first connecting tube 124. The catchment canister 122 is coupled to one end of the two first connecting tubes 124 respectively by the first switch 125, and the other ends of the two first connecting tubes 124 are coupled to the output tube 126. At this time, the first switch 125 can be a one-to-two gas tube switch. Furthermore, the first switch 125 and the output tube 126 may have a containing space respectively matching the first connecting tube 124, so that both ends of the first connecting tube 124 can be fixed between the first switch 125 and the output tube 126 after being inserted into the containing spaces of the first switch 125 and the output tube 126 respectively. In practice, the both ends of the first connecting tube 124 can be fixed between the first switch 125 and the output tube 126 by being totally inserted into the containing spaces of the first switch 125 and the output tube 126 matched with the first connecting tube 124. Because the breathing tube system 12 includes the two first connecting tubes 124, the breathing tube system 12 can further includes a girdle 127 for the user to wear the breathing tube system 12. Moreover, the flame arrester 123 can be configured between the catchment canister 122 and the first switch 125. More specifically, the catchment canister 122 can be coupled to the first switch 125 by the flame arrester 123, or the flame arrester 123 can be contained in the first switch 125 and the first switch 125 is coupled to the catchment canister 122.

However, the catchment canister 122 is configured to collect the liquid in the supply pipeline, and the catchment canister 122 is not necessary to be a part of the supply pipeline. In one embodiment, the catchment canister 122 is coupled to the supply pipeline and configured to collect the liquid in the supply pipeline. As shown in FIG. 4, the breathing gas received by the gas receiving tube 121 flows in sequence from the first switch 125, through the first connecting tube 124 and the flame arrester 123, to the output tube 126. The catchment canister 122 is coupled to the first switch 125 to collect the liquid in the supply pipeline. At this time, the catchment canister 122 does not belong to a part of the supply pipeline. The catchment canister 122 can be configured below the supply pipeline to collect the liquid in the supply pipeline by gravity.

In one embodiment, the maximum inner diameter of the catchment canister 122 is greater than the diameter of the supply pipeline. More specifically, the maximum inner diameter of the catchment canister 122 is greater than the diameter of the gas receiving tube 121 which inputs the breathing gas to the catchment canister 122 and the diameter of the first connecting tube 124 which receives the breathing gas from the catchment canister 122. Since the breathing gas flows from the smaller diameter of tube into the greater inner diameter of the catchment canister 122, the liquid of the breathing gas can be easily left in the catchment canister 122. In detail, as shown in FIG. 7, if the diameter of the gas receiving tube 121 is 6 mm, and each diameter of the first connecting tube 124 is 3 mm. Because the diameters of the gas receiving tube 121 and the first connecting tube 124 are smaller, the gas receiving tube 121 and the first connecting tube 124 are more likely to cause capillary phenomenon, so that the liquid water flows to the output pipe 126 and is inhaled by the user. Therefore, the catchment canister 122 with greater inner diameter is configured between the gas receiving tube 121 and the first connecting tube 124 to keep the liquid left in the supply pipeline, thereby reducing or avoiding the possibility of liquid flowing to the output pipe 126.

Please refer to FIG. 1. The breathing equipment 1 of the present invention further includes a gas generating device 14 coupled to the breathing tube system 12 and generate the breathing gas required by the breathing tube system 12. Wherein, the gas generating device 14 may includes an electrolytic module 142 configured to electrolyze water and generate the aforementioned breathing gas. The mentioned breathing gas is gas comprising hydrogen. Furthermore, the electrolytic module 142 can be contained in a water tank 141 which provides the water to be electrolyzed. Meanwhile, the gas generated by the electrolytic module can be inputted to the water tank 141, and then outputted from the water tank 141 to the breathing tube system 12. However, the gas generated by the electrolytic module 142 may comprise impurities harmful to human body. In one embodiment, the gas generating device 14 further includes a condensing filter 144 configured to condense and filter the gas generated by the electrolytic module 142 to form the mentioned breathing gas. On the other hand, the source gas generated or received by the gas generating device 14 may be too dry and then harmful to human inhalation. Therefore, the gas generating device 14 can include a humidifier 146 configured to humidify the source gas with water to form the mentioned breathing gas.

In addition, the mentioned source gas also can be inhaled by the user together with an atomized gas. In other words, the source gas can be mixed with the atomized gas to form the mentioned breathing gas. Wherein, the atomized gas is generated by the atomizer 148 of the gas generating device 14, and the atomized gas is mixed with the source gas in the atomizer 148 to form the breathing gas received by the breathing tube system 12. In practice, the atomized gas can be formed from an atomizing essential oil or an atomizing portion, so that the breathing gas can further provide a therapeutic effect. The atomizing gas also can be water vapor, which can increase the humidity of the source gas, gas comprising hydrogen or breathing gas to make them suitable for human inhalation. Furthermore, the atomizer 148 can form the aforementioned atomizing essential oil, atomizing portion or water vapor by oscillating the essential oil, portion or water by an oscillator and an oscillating base fluid. Wherein, the mentioned oscillating base fluid can be water.

However, it should be noted that the present invention is not limited to the embodiment that the gas generating device 14 has the electrolytic module 142, the water tank 141, the condensing filter 144, the humidifier 146 and the atomizer 148 at the same time. One or more aforementioned elements can be included in the gas generating device 14. Moreover, the arrangement (sequence, configuration) of the aforementioned elements would not be limited to a specific configuration in the present invention. For example, the condensing filter 144 can be configured after the atomizer 148 to condense the excess water vapor from of the outputted gas, thereby generating the breathing gas.

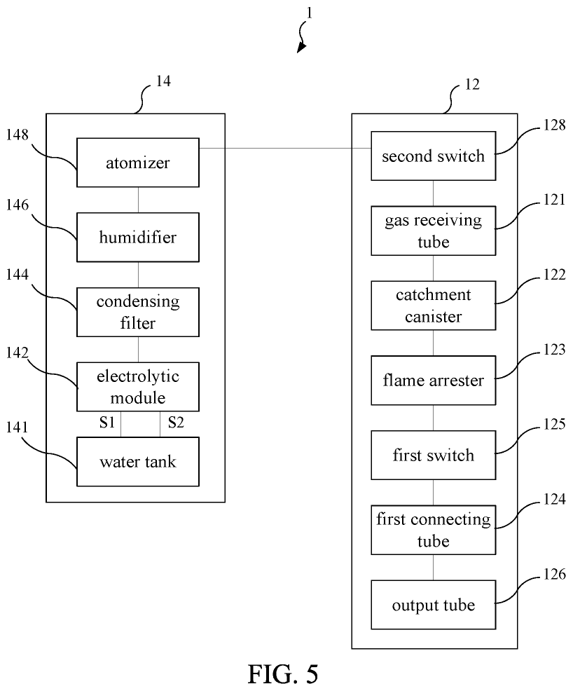

Please refer to FIG. 1 and FIG. 5. FIG. 5 is a function block diagram illustrating the breathing equipment 1 in another embodiment of the present invention. As shown in FIG. 1, in practice, the water tank 141 can contain water and the electrolytic module 142 at the same time, thus the electrolytic module 142 can electrolyze water to generate gas comprising hydrogen and input the gas comprising hydrogen to the water tank 141. However, the present invention is not limited to the embodiment. As shown in FIG. 5, the water tank 141 is configured to contain water and coupled to the electrolytic module 142. The electrolytic module 142 is configured to electrolyze water from the water tank 141 and output the gas comprising hydrogen directly without outputting to the water tank 141. Furthermore, the aforementioned electrolytic module 142 can be the traditional electrolytic cell or the ion-membrane electrolytic cell. Since the cathode and anode of the traditional electrolytic cell are configured in the same space, the gas generated by the cathode and anode will be mixed in the same space when the electrolytic cell electrolyzes water. At this time, the gas comprising hydrogen also includes the oxygen generated by the anode. On the other hand, since the cathode and anode of the ion-membrane electrolytic cell are configured in two different spaces by the ion-membrane, the ion-membrane electrolytic cell can output hydrogen gas and oxygen gas respectively. In other words, the gas comprising hydrogen generated by the ion-membrane electrolytic cell can be pure hydrogen gas. Moreover, please refer to FIG. 5, the electrolytic module 142 can be coupled to the water tank 141 by a first channel S1 and a second channel S2 respectively. When the mentioned electrolytic module 142 is the ion-membrane electrolytic cell, the first channel S1 can be configured to input the water in the water tank 141 to the electrolytic module 142, and the second channel S2 can be configured to input the oxygen gas and excess water generated by the electrolytic module 142 to the water tank 141. At this time, the electrolytic module 142 outputs the gas comprising hydrogen by another channel rather than the first channel S1 and the second channel S2.

As mentioned above, the water tank 141, the humidifier 146 and the atomizer 148 all require liquid (such as water), and the catchment canister 122 of the breathing tube system 12 has a function of collecting liquid. In one embodiment, the catchment canister 122 is further coupled to any one or more of the water tank 141, the humidifier 146 and the atomizer 148 to provide water to the water tank 141, the humidifier 146 or the atomizer 148. In other words, the liquid collected by the catchment canister 122 can be provided for the gas generating device 14. Furthermore, since the liquid collected by the catchment canister 122 may be brought out by the breathing gas outputted by the gas generating device 14, it means that the breathing equipment of the present invention has the function of recycling and improving the use efficiency when the liquid of the catchment canister 122 is provided for the components in the gas generating device 14.

Moreover, the condensing filter 144 may contain the filtered impurities from the breathing gas. In order to prevent the gas channel of the condensing filter 144 from being stuck by the impurities, the condensing filter 144 needs to be periodically cleaned to remove impurities. In one embodiment, the condensing filter 144 can receive an external liquid to remove the impurities from the condensing filter 144. In addition, the impurities may be the electrolytes contained in water when the electrolytic module 142 electrolyze water. In order to improve the use efficiency of the water for electrolyzing, the condensing filter 144 can receive the external liquid to input the impurities back to the electrolytic module 142 or the water tank 141. Furthermore, the above-mentioned liquid can be an additional liquid or a liquid provided from the water tank 141, the humidifier 146 or the atomizer 148. Moreover, all of the mentioned additional liquid, the liquid provided from the water tank 141, the humidifier 146 or the atomizer 148 can include the liquid collected by the catchment canister 122. In other words, the liquid in the condensing filter 144 to clean the channel can be provided from the catchment canister 122.

In summary, the flame arrester is provided in the breathing tube system of the present invention to reduce the possibility of gas ignition to hurt the user. On the other hand, the catchment canister is provided in the breathing tube system of the present invention to collect the liquid in the supply pipeline, thereby reducing the discomfort of the user while inhaling breathing gas. Furthermore, the liquid in the catchment canister can be used to assist in generating breathing gas or to clean the gas pipeline of the gas generating device. Compared with the prior art, the present invention has the advantages of increasing the safety and the comfort of the user, and improving the efficiency of the equipment.

With the examples and explanations mentioned above, the features and spirits of the invention are hopefully well described. More importantly, the present invention is not limited to the embodiment described herein. Accordingly, the above disclosure should be construed as limited only by the metes and bounds of the appended claims.

What is claimed is:

1. A wearable breathing tube system, comprising:
a gas receiving tube configured to receive a breathing gas;
a first connecting tube coupled to the gas receiving tube;
an output tube coupled to the first connecting tube, the output tube being configured for a user to wear and to output the breathing gas to the user, wherein the gas receiving tube, the first connecting tube and the output tube form a supply pipeline; and
a flame arrester configured in the supply pipeline.

2. The system of claim 1, further comprising a catchment canister coupled to the gas receiving tube and the first connecting tube for collecting a liquid in the supply pipeline, wherein the diameters of the gas receiving tube and the first connecting tube are smaller than the maximum inner diameter of the catchment canister.

3. The system of claim 2, further comprising a first switch coupled to the first connecting tube, the catchment canister being separably coupled to the gas receiving tube and the first switch.

4. The system of claim 2, wherein the catchment canister has a first canister body and a second canister body which can be separated from each other, and a catchment canister sealing ring is configured between the first canister body and the second canister body to couple to the first canister body and the second canister body.

5. The system of claim 2, wherein the flame arrester is configured between the catchment canister and the first connecting tube.

6. The system of claim 5, further comprising a first switch coupled to the first connecting tube and the catchment canister, the flame arrester being contained in the first switch and coupled to the catchment canister by the first switch.

7. The system of claim 6, wherein the output tube has two gas outlets to output the breathing gas to the user, the first connecting tube comprises a pair of sub-connecting tubes respectively coupled to a side opening of the output tube.

8. A breathing equipment, comprising:
a wearable breathing tube system, comprising:
a gas receiving tube configured to receive a breathing gas;
a first connecting tube coupled to the gas receiving tube;
an output tube coupled to the first connecting tube, the output tube being configured for a user to wear and to output the breathing gas to the user, wherein the gas receiving tube, the first connecting tube and the output tube form a supply pipeline; and
a catchment canister coupled to the supply pipeline for collecting a liquid in the supply pipeline;
wherein, the diameter of the supply pipeline is smaller than the maximum inner diameter of the catchment canister.

9. The equipment of claim 8, wherein the breathing tube system further comprises a first switch configured to couple to the first connecting tube and the catchment canister.

10. The equipment of claim 9, wherein the first connecting tube comprises a pair of sub-connecting tubes, the sub-connecting tubes are coupled to the catchment canister by the first switch.

11. The equipment of claim 8, wherein the catchment canister is configured between the gas receiving tube and the first connecting tube, the breathing tube system further comprises a flame arrester configured between the catchment canister and the first connecting tube.

12. The equipment of the claim 8, further comprising an electrolytic module configured to electrolyze the liquid and generate the breathing gas.

13. The equipment of claim 8, further comprising an atomizer configured to generate an atomizing gas from the liquid and to mix the atomizing gas with a source gas to form the breathing gas.
